(19) United States
(12) Patent Application Publication
(10) Pub. No.: US 2021/0275825 A1
(43) Pub. Date: Sep. 9, 2021

(54) RF DEVICE FOR SKIN AND FAT TREATMENT

(71) Applicant: Inmode Ltd., Yokneam (IL)

(72) Inventor: Michael Kreindel, Richmond Hill (CA)

(73) Assignee: Inmode Ltd., Yokneam (IL)

(21) Appl. No.: 17/327,980

(22) Filed: May 24, 2021

Related U.S. Application Data
(63) Continuation of application No. 15/954,930, filed on Apr. 17, 2018.

(51) Int. Cl. A61N 1/40

(52) U.S. Cl. CPC A61N 1/403

(57) ABSTRACT

The invention relates to a radio-frequency (RF) device for non-ablative treatment of skin and subcutaneous fat using applicator with multiple RF delivering elements.

### CROSS-REFERENCE TO OTHER APPLICATIONS

This application claims priority from, and is a continuation of, U.S. patent application Ser. No. 15/954,930, filed 17 Apr. 2018, which is incorporated herein by reference.

### FIELD OF THE INVENTION

The invention relates to a radio-frequency (RF) device for non-ablative treatment of skin and subcutaneous fat using applicator with multiple RF delivering elements.

### BACKGROUND OF THE INVENTION

RF devices for non-ablative treatment have become common place in medical aesthetic practice. The devices are differentiated by type of energy application. Some of the treatments are based on moving a hand piece over the skin surface and deliver RF energy continuously. The treatment effect is achieved by elevating skin temperature during multiple passes. RF energy is delivered in continuous mode and the hand piece is moved over the treated area to attain the skin target temperature and maintain it for the required time.

The other broadly used treatment option is application of all the energy required for the treatment effect at one spot and then moving the hand piece to a new spot and repeating the procedure. Bi-polar and mono-polar RF devices are used for this type of treatment. Typically, the tissue is heated to 40-50° C. and maintained over a period of time varying from a few minutes up to 30 minutes. Skin temperature can be monitored to optimize the treatment.

Treatment results are a function of the heating level, which is limited by safety and comfort levels and time that heating is maintained. Long treatment is annoying for the treatment attendant and expensive for the patient.

### SUMMARY OF THE INVENTION

The present invention is a device for delivering RF energy to the treatment zone with minimal involvement of a treatment attendant. The device has a platform connected to one or more applicators. Each applicator comprises multiple identical RF elements coupled to the skin. RF energy is applied (simultaneously or not) by the multiple elements so as to reach and maintain a required tissue temperature during a predetermined period of time. The amount of RF elements depends on the treatment zone.

The applicator can connect the multiple elements rigidly or flexibly, such as with a flexible material, e.g., a felt or silicone belt or pad.

Each element in the device may have an identical geometry of electrodes and distance between them. The electrodes in each element are isolated from one another to avoid RF current exchange between elements. Mono-polar or bi-polar RF technology can be used, but for treatment of a large area the bi-polar system is preferable to prevent thermal effect near the return electrode.

Each element may have one or more electrodes. Alternatively, there may be an array of electrodes with equal distance between them, and RF energy is applied continuously or intermittently between each two adjacent electrodes. In this case, each group of electrodes participating in RF energy delivery is considered as an element.

In one embodiment the RF energy from the one RF generator is switched continuously or intermittently between elements to create a heating effect in the treated tissue. Alternatively, RF energy can be delivered to the multiple elements simultaneously from independent RF generators.

Tissue impedance and RF parameters can be monitored by the system for each element and the RF energy can be adjusted according to feedback from these measurements. If measured impedance is out of an acceptable range, the RF energy can be stopped.

With a large area of electrodes and good coupling, the RF energy can be delivered directly from the electrodes to the skin. Alternatively, gel or conductive pads can be used for coupling between electrodes and the treated tissue.

In some embodiments, a negative pressure can be used for coupling skin to the RF electrode located in a cavity connected to a vacuum pump.

A temperature sensor can be embedded in each element to control the heating process. RF energy can be adjusted for each element independently according to feedback from the corresponding temperature sensor. The temperature sensor can be a thermistor, thermocouple, optical, or other.

The typical average RF energy density delivered from the element is up to 5 W/cm². RF energy can be reduced when target temperature is approached. RF energy can be switched on and off to maintain target temperature for the predetermined treatment time. The treatment time is variable, such as without limitation, from 10 min and up to 120 min. The target temperature is variable, such as without limitation, from 40° C. to 50° C. RF frequency may be, without limitation, in the range of 100 kHz up to 40 MHz. The preferable range is 400 kHz to 6 MHz.

This type of treatment can be applied to collagen remodeling, wrinkle reduction, skin tightening, fat destruction, cellulite treatment, and other treatments where bulk tissue treatment is required.

The system can be used for treatment of the face, neck, or other body areas. Shape of the applicator and the number of elements depend on the treatment area. More than one applicator can be connected to the RF generator. Applicators intended for body treatment may have larger elements to cover larger area and for deeper RF energy penetration while facial or neck applicators may have smaller elements.

### BRIEF DESCRIPTION OF THE DRAWINGS

In the accompanying drawings:

FIG. 1 is a schematic illustration of one example of applicator applied to the patient face.

FIG. 2 is a schematic illustration of the applicator with multiple elements.

FIG. 3 is a schematic illustration of one example of single element having three RF electrodes.

FIG. 4 is a schematic illustration of one example of single element having two RF electrodes.

### DETAILED DESCRIPTION OF THE INVENTION

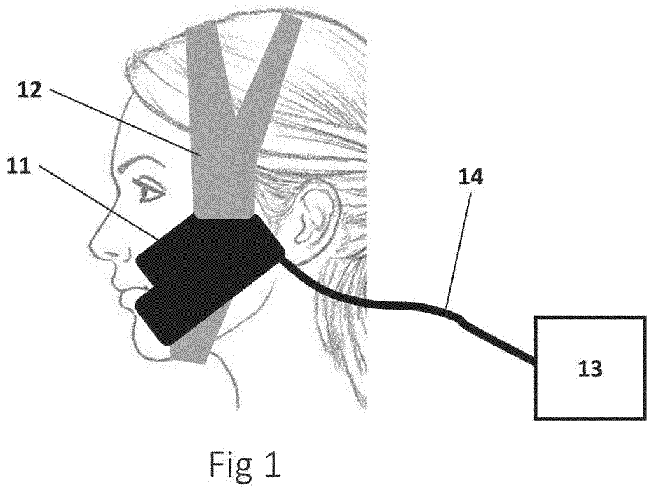

Referring first to FIG. 1, an applicator 11 is applied to the patient face and secured with belt 12. The applicator 11 is connected to an RF generator 13 through a cable or belt 14. The belt 14 may be made from an elastic material and can be adjusted for an individual patient. RF generator 13 supplies RF energy to each element in applicator 11 and processes the feedback from impedance and temperature measurements. The treatment attendant can set individual parameters for each patient or treatment zone. In addition to or instead of facial applicator 11, other applicators designed for different treatment area can be connected to the device. Another symmetrical applicator may be connected to the opposite side of the face and also connected to the RF generator 13.

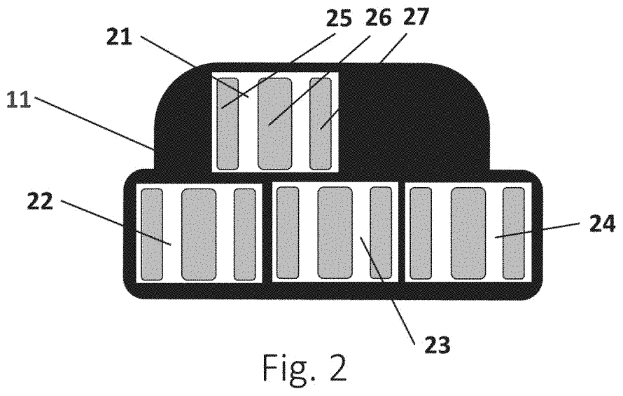

FIG. 2 shows the facial applicator comprising four identical RF elements 21, 22, 23, 24 delivering RF energy to the treated skin area. Each element has three RF electrodes 25, 26, 27, wherein central electrode 26 has one polarity and side electrodes 25, 27 have opposite polarity. Each element is isolated electrically from other elements.

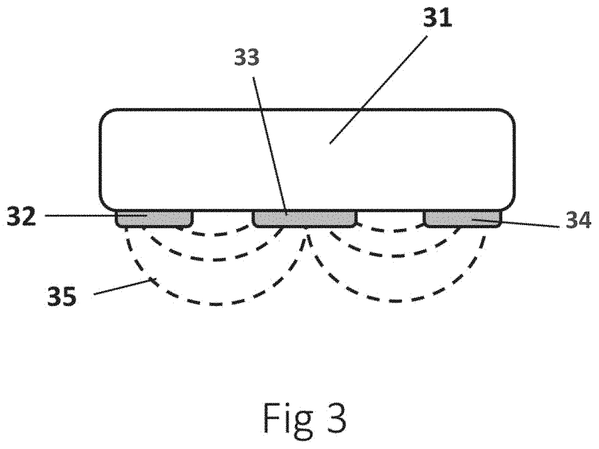

FIG. 3 shows schematically one element 31 having three RF electrodes 32, 33, 34. Central RF electrode 33 has one polarity while side electrodes 32 and 34 have opposite polarity. RF current 35 flows from the central electrode to the side electrodes and RF energy penetration depth depends on the size of the electrodes and the distance between them. The area of central electrode 33 may be similar to the total area of both side electrodes 32, 34.

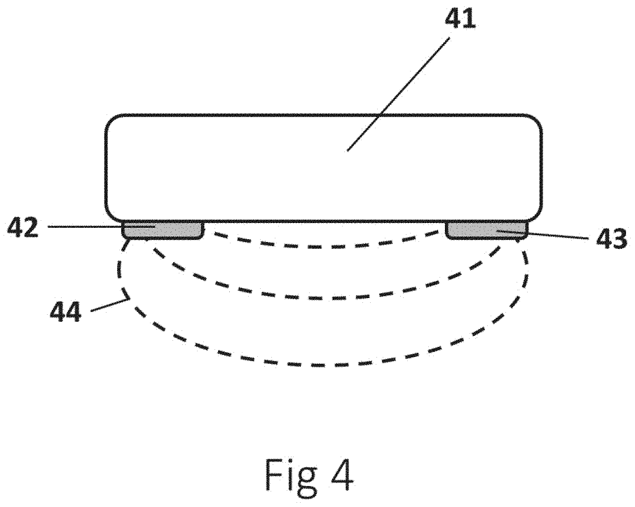

Referring to FIG. 4, an alternative element assembly 41 is shown, wherein RF current 44 flows between two electrodes 42, 43. RF energy distribution in the tissue depends on the size of the electrodes and the distance between them. A larger distance between the electrodes causes a deeper RF energy penetration into the tissue.

Non-limiting parameters for the device are as follows:

1. RF peak voltage applied to the tissue in the range of 10 V up to 1000 V

2. RF frequency in the range of 100 kHz up to 40 MHz

3. Number of elements in applicator depends on treatment area and can be varied from 2 to 20 elements.

4. Each element may have an area of 1 cm² to 7 cm² for treatment of small zones as the face or neck and 6 cm² to 30 cm² for large body areas.

5. Temperature sensor may be embedded into each element to control maximal tissue heating to be in the range of 40° C. to 50° C. (without limitation) according to user setting.

6. RF energy is controlled according to feedback from temperature sensor and impedance measurements. If measured parameters are out of an acceptable range, the RF energy is terminated.

1. A device comprising:
a face applicator comprising a face securing member configured to secure said face applicator to at least one cheek of a subject; and
radio-frequency (RF) elements coupled to said face applicator and coupled to an RF generator; and
impedance and temperature measurement sensors coupled to said RF generator.

2. The device according to claim 1, wherein said face securing member comprises symmetrical portions for securing said face applicator to both cheeks of the subject.

3. The device according to claim 1, wherein said RF elements comprise a central electrode which has a polarity and side electrodes which have a polarity opposite to said polarity of said central electrode.

4. The device according to claim 3, wherein a treatment area of said central electrode is identical to a total treatment area of said side electrodes.
